(19) United States
(12) Patent Application Publication
(10) Pub. No.: US 2018/0120310 A1
(43) Pub. Date: May 3, 2018

(54) Method of and System for Printing In-Well Calibration Features

(71) Applicant: Aushon Biosystems, Inc., Billerica, MA (US)

(72) Inventors: Peter HONKANEN, Concord, MA (US); Christine A. BURNS, Wellesley, MA (US)

(73) Assignee: Aushon Biosystems, Inc., Billerica, MA (US)

(21) Appl. No.: 15/852,939

(22) Filed: Dec. 22, 2017

Related U.S. Application Data
(62) Division of application No. 13/630,917, filed on Sep. 28, 2012.
Application No. 13/630,917 is a continuation of application No. PCT/US11/61184, filed on Nov. 17, 2011.
(60) Provisional application No. 61/414,663, filed on Nov. 17, 2010.

(51) Int. Cl. G01N 33/543; B01L 3/00

(52) U.S. Cl. CPC G01N 33/54393; B01L 3/5085; B01J 2219/00725; B01J 2219/00315; B01L 2200/148; B01L 2300/0636; G01N 33/54366; B01J 2219/00576; B01L 2300/0829; B01J 2219/00693; B01J 2219/0074; B01J 2219/00385

(57) ABSTRACT

An apparatus and a method are disclosed for printing in-well calibration features onto assay substrates. An apparatus includes a testing substrate; a plurality of capture compound features in a well of the testing substrate; a calibration feature on one of the capture compound features in the well of the testing substrate, where the calibration feature has a known concentration of a compound that is capable of binding to the capture compound; and at least one additional capture compound feature in the same well of the testing substrate, where the at least one additional capture compound feature does not have a calibration feature printed onto the at least one additional capture compound feature. Methods for using the same are disclosed.

### CROSS-REFERENCE TO RELATED APPLICATIONS

This application is a divisional of U.S. patent application Ser. No. 13/630,917, filed Sep. 28, 2012, which is a continuation of International Application PCT/US2011/61184, filed Nov. 17, 2011, which claims the benefit under 35 U.S.C. § 119(e) of U.S. Provisional Patent Application Ser. No. 61/414,663, filed on Nov. 17, 2010, all of which are incorporated by reference herein in their entirety.

### BACKGROUND

### Field of Invention

The present invention relates to preparation of assay substrates, and, more specifically, to methods and systems for printing in-well calibration features onto assay substrates.

### Description of Related Art

An assay substrate is a surface upon which various chemical and/or biological analyses can be performed. Examples of an assay substrate include microarray plates, glass slides, and microtiter plates. A microtiter plate is a flat plate that has multiple “wells” formed in its surface. Each well can be used as a small test tube into which various materials can be placed to perform biochemical analyses. One illustrative use of microtiter plates includes an enzyme-linked immunosorbent assay (ELISA), which is a modern medical diagnostic testing technique.

Generally, in an ELISA, a capture antibody is printed in the bottom of a well in a microtiter plate. The capture antibody has specificity for a particular antigen for which the assay is being performed. A sample to be analyzed is added to the well containing the capture antibody, and the capture antibody “captures” or immobilizes the antigen contained in the sample. A detect antibody is then added to the well, which also binds and/or forms a complex with the antigen. Further materials are then added to the well which cause a detectable signal to be produced by the detect antibody. For example, when light of a specific wavelength is shone upon the well, the antigen/antibody complexes will fluoresce. The amount of antigen in the sample can be inferred based on the magnitude of the fluorescence. In another example, a compound can be added to the well that causes the detect antibody to emit light within a predetermined wavelength (e.g., 400-500 nm). This light can be read by a charged-coupled device (CCD) camera to measure the optical brightness of the emitted light.

During an ELISA, the absorbency, fluorescence, or electrochemical signal of the well can be measured and compared with a standard to more accurately determine the presence and quantity of the sample antigen. For example, a calibration feature with a known concentration of antigen can be placed in wells separate from the wells that receive antigen-containing patient samples. However, signal variability, such as fluorescence variability, in the different wells can decrease the accuracy of comparing results from separate wells.

Thus, a need exists for methods and systems to provide and improve accuracy and reliability in medical diagnostic testing techniques and other biochemical analyses.

### BRIEF DESCRIPTION OF THE DRAWINGS

FIGS. 1A-B show a cross-sectional side view and a top view, respectively, of two wells in a microtiter plate.

FIGS. 2A-C show a series of cross-sectional side views of a well in a microtiter plate during a known method of conducting an ELISA.

FIG. 3 shows a method of preparing in-well calibration features in accordance with some embodiments.

FIGS. 4A-C show a series of cross-sectional side views of a well in a microtiter plate during a method of conducting an ELISA in accordance with some embodiments.

FIG. 5 shows a cross-sectional side view of a well in a microtiter plate with a number of printed features in accordance with some embodiments.

### DETAILED DESCRIPTION

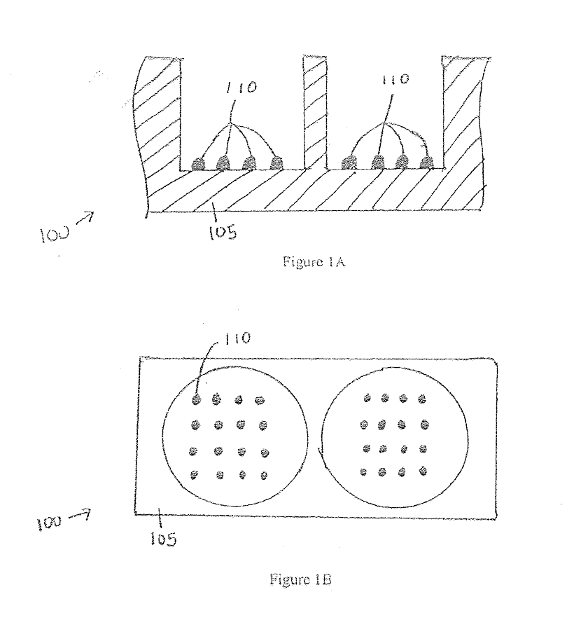

FIG. 1A shows an illustration of a cross-sectional side view of two wells in a microtiter plate 100. In one illustrative implementation, the well substrate is formed of a polystyrene base 105. Other potential substrate materials include, but are not limited to, nitrocellulose, glass, and other plastic materials. FIG. 1B shows an illustration of a top view of two wells in a microtiter plate 100. During the preparation of a microtiter plate for use in biochemical analysis, many different capture antibody “features” 110 are printed in the well and adhere to the polystyrene base 105. As used herein, “features” can have different shapes, such as, for example, a rounded shape. The assay substrate can be, for example, a 96-well microtiter plate. The features can be, for example, about 300 μm to about 500 μm in diameter.

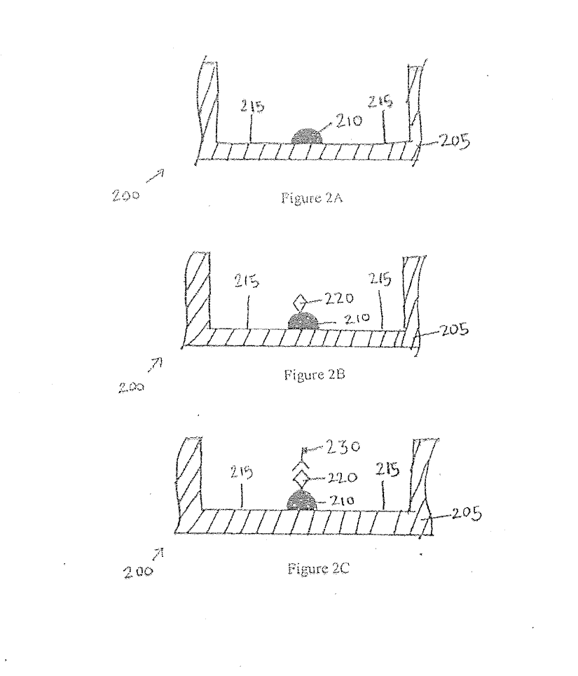

FIGS. 2A-C show a series of cross-sectional side views 200 of a well 205 during a known method of conducting an ELISA. After the capture antibody feature 210 has been printed onto the bottom of the well 205, a blocking material is added to the well to block plate binding sites 215 that remain on the plate 200. This prevents non-selective binding of sample antigens to the base of the well during the ELISA, which would give false readings. Second, an antigen-containing sample is added to the well. FIG. 2B shows the antigen 220 binding to the capture antibody feature 210. Third, the well is washed so that unbound antigen is removed. Fourth, enzyme-linked detect antibodies are added. FIG. 2C shows the enzyme-linked detect antibody 230 binding to the antigen 220. The well is then washed so that unbound antibody-enzyme conjugates are removed. Next, a substance is applied which converts the enzyme into a detectable signal, such as a color, fluorescent, or electrochemical signal. Finally, the absorbency, fluorescence, or electrochemical signal of the well is measured and compared with a standard to determine the presence and quantity of the sample antigen. A standard can be generated by printing calibration features with a known concentration of antigen in wells that are separate from the wells that receive patient samples.

Such an approach involves comparing standard results and sample results from different wells. Signal variability, such as fluorescence variability, and well-to-well variability in the separate wells can decrease the accuracy and reliability of test results.

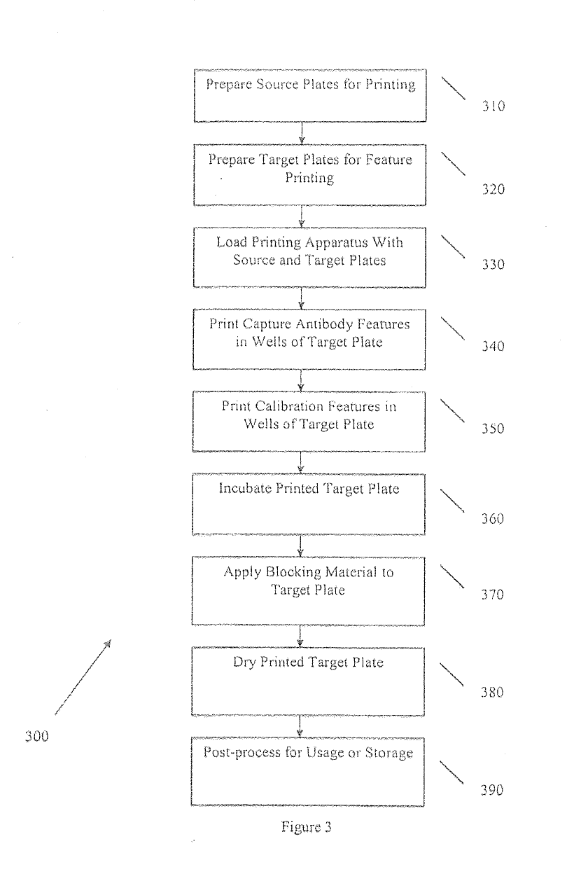

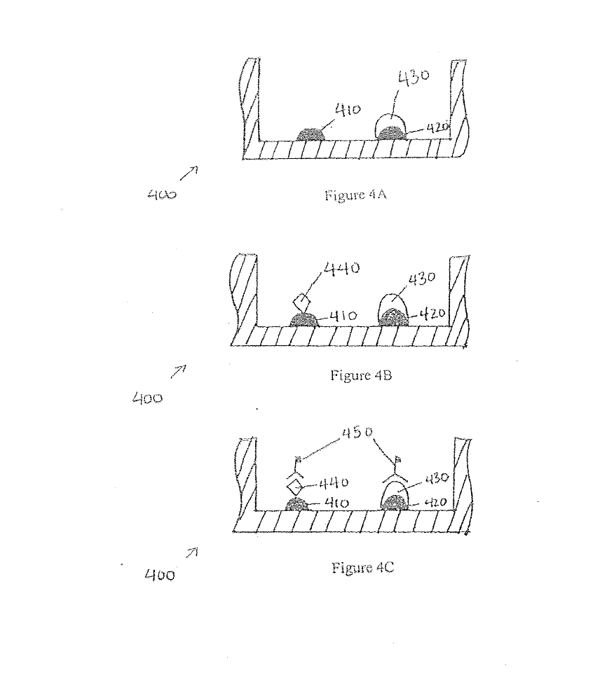

In one illustrative embodiment, FIG. 4A shows a cross-sectional side view of one plate well with two capture antibody features 410 and 420 and a calibration feature 430 printed on top of capture antibody feature 420. The calibration feature 430 is in the same well as the capture antibody feature 410 that will bind to the antigen-containing sample. FIG. 3 shows a method 300 of printing in-well calibration features on a microtiter plate in accordance with some embodiments. Method 300 reduces or eliminates the inaccuracy that can result from signal variability and well-to-well variability by printing a calibration feature with a known amount of antigen in the same well as the capture antibody feature that binds to antigen-containing samples. Not only does method 300 reduce the variance of assay results, it also increases throughput, as all the wells of a plate can be used to analyze patient samples.

Suitable samples include proteomic samples such as, for example, from cell lysates, cell supernatants, plasma, serum, or other biological fluids. As used herein, a “target plate” is a plate that is to be prepared (e.g., printed, blocked, and processed for later usage) for a particular set of analyses. A “source plate” is a plate that has a supply of the material to be printed onto a target plate. For example, the wells of a source plate can be filled with various types of antibodies that are to be printed onto target plates. In accordance with method 300, the source plate is prepared for the printing process (step 310). This can include filling the wells of the source plate with the desired material to be printed onto the target plate. Next, the target plate is prepared for printing (step 320). This can include washing and/or performing other surface treatments to enable the material to be printed to properly adhere to the bottom surface of the plate well.

The source and target plates are then fit into a printing apparatus (e.g., a 2470 Arrayer available from Aushon Biosystems, Inc. of Billerica, Mass.) (step 330). Capture antibody features are printed in the wells of the target plate (step 340). Next, calibration features with a known concentration of an antigen are precisely printed onto the capture antibody features (step 350). The known concentration of an antigen ranges from the order of femtogram (10⁻¹⁵ g) per milliliter to milligram (10⁻³ g) per milliliter. Implementations of the invention using the 2470 Arrayer, independent of the arrayer's pin size, can achieve precise printing of the calibration features onto the capture antibody features, such that the positional misalignment between an outer edge of the calibration features and an outer edge of the capture antibody features is about 4 μm or less. Other implementations of the invention can tolerate positional misalignments between an outer edge of the calibration features and an outer edge of the capture antibody features of greater than about 4 μm, depending on the size of the features. For example, when printing features are in the range of about 120 μm to about 240 μm in diameter, positional misalignment between an outer edge of the calibration features and an outer edge of the capture antibody features of about 10 μm can be tolerated.

As described above, FIG. 4A shows a cross-sectional side view of one plate well with two capture antibody features 410 and 420 and a calibration feature 430 printed on top of capture antibody feature 420. The calibration feature 430 is in the same well as the capture antibody feature 410 that will bind to the antigen-containing sample.

The printed target plate is incubated for a period of time (step 360), and a blocking material, which does not react to the capture antibody, is applied to the target plate using known methods (step 370). The blocking material adsorbs to the remaining binding surfaces of the plate and binds to antigens of non-specific interaction, thus reducing background signal. The printed target plate is then dried (step 380). In one illustrative implementation, a blocking material solution is applied to the surfaces of the bottoms of a plurality of wells in a microtiter plate via a spraying process, as described in U.S. Provisional Patent Application 61/372,552 entitled Method of and System for Applying Blocking Material to Assay Substrates, filed on Aug. 11, 2010, the contents of which are incorporated by reference in its entirety.

During the spraying process, an airbrush (e.g., a Paasche Talon model TG0210) is used to apply the blocking material to the bottom surface of the well of the plate. During the spraying step, approximately 10 ml of a blocking material solution is sprayed over the entire surface of the plate. The blocking material is propelled by a compressed air source, e.g., a standard air compressor that supplies clean and dry air, at a pressure of about 138 kPa (20 psig). The flow rate of the airbrush is set to about 10 ml/min. The application of the blocking material reduces or eliminates malformation and/or toppling of features during the addition of blocking material to the microtiter wells. The plates prepared according to the spraying process discussed herein have superior feature uniformity

In some embodiments, the nozzle of the airbrush is positioned about 15 cm (6 inches) from the surface of the plate, and the airbrush is swept across the entire surface while keeping the nozzle perpendicular to the surface of the plate. In other words, the center of the spray pattern is essentially normal to the surface of the plate. The spraying is continued at least until the level of blocking material in the well covers the printed features 530. After that level of blocking material is achieved, additional blocking material can be added by continuing the spraying process, or, optionally, additional blocking material can be added via micropipette, as described herein.

The application of the blocking material as described herein can be applied by-hand. In some implementations, the blocking can be applied by automated machinery. For example, after printing, incubating, and drying, the plate can be placed on a conveyor over which is mounted one or more spray nozzles. The rate of the conveyor is controlled to ensure adequate residence time of the plates within the spray pattern of the one or more nozzles. For example, if the total flow rate of all of the nozzles is about 10 ml/min, the conveyor speed can be controlled to provide that at least some portion of the surface of the plate is under the spray pattern for 1 minute. In another illustrative implementation, the plate can be held is a fixed position and an automated arm can direct one or more spray nozzles above the surface of the plate.

The specific operational parameters provided herein are merely illustrative, and other values are within the scope of the invention. For example, the blocking material flow rate can vary between 5-20 ml/min, the distance between the airbrush flow nozzle and the surface of the plate can vary between 2-41 cm (1-16 inches), and the air pressure can vary between 34-207 kPa (5-30 psig). It is understood that these ranges are merely illustrative and are not intended to be limiting.

The target plate is then processed for usage or storage using known methods (step 390). For example, the target plate can be incubated at about 4° C. overnight. Alternatively, excess blocking material (e.g., the blocking material that has not bound to the bottom of the well) can be removed from the target plate, the plate can then be dried, and then the plate can be placed into a moisture-resistant package for storage. The disclosed method of printing in-well calibration features reduces or eliminates inaccuracy that can result from having the calibration feature printed in a separate well from the capture antibody feature that will bind to the antigen-containing sample. The disclosed method also increases throughput, as all the wells of a plate can be used to analyze patient samples.

The plates with in-well calibration features can then be used to conduct chemical and/or biological analyses, such as with an ELISA. FIG. 4B shows a cross-sectional side view of the well after an antigen-containing sample has been added, and patient antigen 440 binds to the capture antibody 410. Next, enzyme-linked detect antibodies are added to the well. FIG. 4C shows the enzyme-linked detect antibody 450 binding to the antigen 440 and calibration feature 430. A substance, such as a chemiluminescent substrate solution, is applied to convert the enzyme into a detectable signal. Finally, the signals are measured, and the presence and quantity of the sample antigen is determined using methods known in the art.

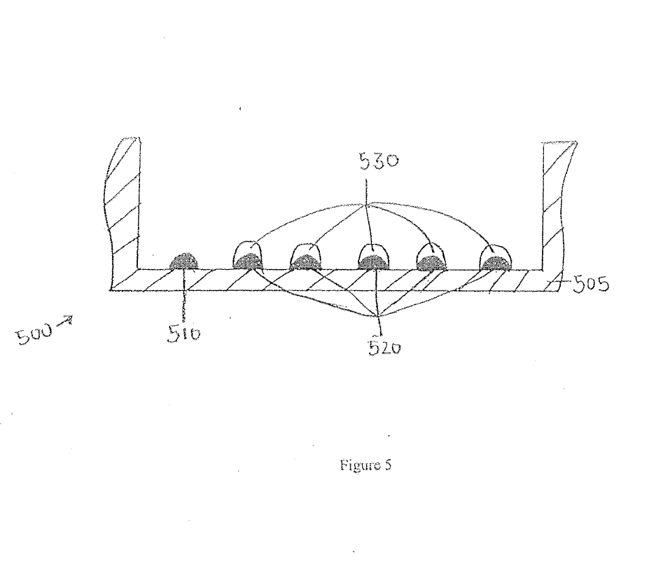

In another illustrative embodiment, two or more capture antibody features can be printed on each well, and one or more calibration features of varying antigen concentrations can be printed on each well. FIG. 5 shows a cross-sectional side view of one plate well with capture antibody features 510 and 520 printed at the bottom of the well 505. Five calibration features 530 with varying concentrations of antigen are precisely printed on top of capture antibody features 520. The series of calibration features with varying concentrations of antigen can be used to generate a standard curve. With the varying concentrations of the calibration features being known, the features produce detectable signals of varying intensity related to the known concentrations. TWC 2204751-00120 Mitchell Articlehe standard curve can be compared to the signal of the capture antibody feature binding to the antigen-containing test sample to determine the presence and quantity of the sample antigen. The disclosed method reduces or eliminates inaccuracy that can result from having the series of calibration features printed in separate wells from the capture antibody feature that will bind to the antigen-containing sample. It also results in increased throughput and efficiency of assays and other analyses.

The specific operational parameters provided above are merely illustrative, and other values are within the scope of the invention.

Kits can be made that incorporate the above devices along with any combination of related equipment or reagents, such as reporter reagents or software for reading results of the assay.

The embodiments described above can be used to detect the presence of antigens and proteins in a patient, such as a patient having an autoimmune disease, antibodies to viral diseases, antibodies to bacterial diseases, antibodies to allergic reactions, or antibodies to cancers.

The terms and expressions that are employed herein are terms of description and not of limitation. There is no intention in the use of such terms and expressions of excluding the equivalents of the feature shown or described, or portions thereof, it being recognized that various modifications are possible within the scope of the invention as claimed.

1. A method comprising:
printing a plurality of capture compound features in a well of a testing substrate;
printing a calibration feature on one of the capture compound features in the well of the testing substrate;
wherein the calibration feature has a known concentration of a compound that is capable of binding to the capture compound feature; and
wherein at least one capture compound feature does not have a calibration feature printed thereon.

2. The method of claim 1, further comprising printing a plurality of calibration features on a respective plurality of capture compound features, wherein the plurality of calibration features includes at least two different concentrations of the compound that is capable of binding to the capture compound.

3. The method of claim 1, further comprising:
incubating the printed testing substrate;
applying blocking material to the testing substrate;
drying the printed testing substrate; and
processing the printed testing substrate for usage or storage.

4. The method of claim 1, wherein the testing substrate is used to conduct biochemical analyses.

5. The method of claim 1, wherein capture compound feature is a capture antibody feature, and the compound that is capable of binding to the capture antibody feature is an antigen.

6. The method of claim 4, wherein the biochemical analysis is an enzyme-linked immunosorbent assay.

7. The method of claim 2, further comprising:
using the results from the at least two different concentrations of the compound that is capable of binding to the capture compound to create a calibration curve;
comparing the calibration curve to a signal of a capture compound feature binding to an antigen-containing test sample; and
determining the presence and quantity of the antigen in the test sample.

8. The method of claim 1, wherein the misalignment between an outer edge of the calibration feature and an outer edge of the capture compound feature is about 10 μm or less.

9. The method of claim 1, wherein the calibration feature has a diameter of between about 120 μm to about 500 μm.

10. The method of claim 1, wherein the capture compound feature has a diameter of between about 120 μm to about 500 μm.

11. The method of claim 1, further comprising applying a blocking material to at least a portion of the well that does not contain a capture compound feature thereon.

12. The method of claim 1, further comprising determining the presence or quantity of a compound on the at least one of the plurality of capture compound features that does not have a calibration feature printed thereon.

13. The method of claim 12, wherein determining the presence or quantity of a compound further comprises conducting an ELISA.

14. The method of claim 12, wherein determining the presence or quantity of a compound further comprises comparing a signal from the at least one capture compound feature that does not have a calibration feature printed thereon to a calibration curve derived from the calibration feature.

15. The method of claim 12, wherein the compound on the at least one capture compound feature that does not have a calibration feature printed thereon is the same as the compound capable of binding to the capture compound.

16. The method of claim 1, wherein the substrate comprises a plurality of wells.

17. The method of claim 16, wherein each of the wells is used to analyze a sample.

18. The method of claim 16, further comprising:
printing a plurality of capture compound features in at least two wells of the testing substrate; and
printing a calibration feature on at least one of the capture compound features in at least two wells of the testing substrate;
wherein the calibration features have at least one known concentration of a compound that is capable of binding to at least one of the capture compound features; and
wherein at least one of the capture compound features in at least two wells does not have a calibration feature printed thereon.

19. The method of claim 18, further comprising identifying the presence or quantity of a compound on the at least one of the capture compound features in at least two wells that does not have a calibration feature printed thereon, and wherein the identified compound is the same as the compound that is capable of binding to at least one of the capture compound features.

20. The method of claim 1, wherein the known concentration of a compound capable of binding to the capture compound feature has a concentration between a femtogram per milliliter to a milligram per milliliter.
